(19) United States
(12) Patent Application Publication
(10) Pub. No.: US 2026/0139218 A1
(43) Pub. Date: May 21, 2026

(54) PREPARATION METHOD FOR HIGH-ACTIVITY FREEZE-DRIED BIFIDOBACTERIUM PREPARATION BASED ON PH REGULATION, AND APPLICATION

(71) Applicant: Inner Mongolia Agricultural University, Hothot (CN)

(72) Inventors: Guoqiang YAO, Hothot (CN); Heping ZHANG, Hothot (CN); Kailong LIU, Hothot (CN); Wenjun LIU, Hothot (CN); Wenyi ZHANG, Hothot (CN); Yongfu CHEN, Hothot (CN); Zhihong SUN, Hothot (CN)

(73) Assignee: Inner Mongolia Agricultural University, Hothot, NM (CN)

(21) Appl. No.: 19/339,271

(22) Filed: Sep. 24, 2025

(51) Int. Cl. C12N 1/04; C12N 1/205; C12Q 1/02; C12R 1/01

(52) U.S. Cl. CPC C12N 1/04; C12N 1/205; C12Q 1/02; C12R 2001/01

(57) ABSTRACT

The present disclosure relates to the field of biotechnology, and in particular to a preparation method for a high-activity freeze-dried Bifidobacterium preparation based on pH regulation, and an application. According to the present disclosure, by determining the influence of pH on the growth activity of the Bifidobacterium breve during culture and the influence on the survival rate of the freeze-dried bacterial cells, it is found that low pH can improve the resistance ability of the bacterial cells to freeze-drying and storage environment stress, which provides a basis for preparing the high-activity freeze-dried Bifidobacterium preparation.

### TECHNICAL FIELD

The present disclosure relates to the field of biotechnology, and in particular to a preparation method for a high-activity freeze-dried Bifidobacterium preparation based on pH regulation, and an application.

### BACKGROUND

pH regulation is an important regulation means and basic link in fermentation and production of probiotics, which aims at regulating division of bacterial cells by regulating pH of a fermentation system, forming an environment conducive to rapid division and proliferation of the bacterial cells, realizing precise fermentation and improving processing activity of probiotic preparations.

Bifidobacterium, as an important probiotic, faces the problems of low biomass and potential loss of activity in industrial production. Although pH regulation is widely applied to probiotic fermentation, research on a molecular mechanism of using pH to regulate growth of Bifidobacterium is still insufficient.

Vacuum freeze drying technology is a common method for producing probiotic preparations. However, bacterial cells are likely to be damaged or even killed during freeze drying, and survival rate, cell activity, cell membrane function and intracellular enzyme activity all can decrease after freeze drying. Therefore, pH regulation is used for reducing the degree of cell damage in a fermentation process. pH regulation is an important means and basic link in the production and processing process of the probiotics, and aims at improving the freeze-drying activity and storage stress resistance of the bacterial cells through proper regulation.

### SUMMARY

An objective of the present disclosure is to provide a preparation method for a high-activity freeze-dried Bifidobacterium preparation based on pH regulation, and an application. Through dynamic pH regulation and culture, the Bifidobacterium preparation with higher bacterial cell activity and storage stress resistance is developed.

In order to achieve the above objective, the present disclosure provides a preparation method for a high-activity freeze-dried Bifidobacterium preparation based on pH regulation. The preparation method includes the following steps:
S1. activating a strain of Bifidobacterium breve to obtain a third generation seed solution, inoculating the third generation seed solution in a modified MRS culture medium, and culturing the Bifidobacterium breve through dynamic pH regulation;
S2. testing growth activity of bacterial cells under different pH and different growth phases; and
S3. freeze-drying the cultured bacterial cells to obtain a freeze-dried preparation, determining a survival rate of bacterial cells in the freeze-dried preparation, and screening out a culture system with the highest survival rate of bacterial cells.

In the present disclosure, an activation temperature in S1 is 36-38° C., and activation time is 23-25 h.

In the present disclosure, different growth phases in S2 include a logarithmic phase, a stationary phase and a death phase.

In the present disclosure, the testing growth activity of bacterial cells under different pH and different growth phases includes testing a viable count, a density, a maximum specific growth rate and bacterial cell area distribution of the bacterial cells under different pH and different growth phases.

A formula for calculating the maximum specific growth rate is:

$$y = \frac{a}{1 + \frac{( a - {y}_{0} ) \times {e}^{- 4 Wmax \frac{x}{a}}}{{y}_{0}}} ,$$

in the formula, x is culture time, y is a bacterial cell density, y₀ is an initial bacterial cell density, a is a maximum bacterial cell density, and Wₘₐₓ is the maximum specific growth rate.

In the present disclosure, a freeze-drying process in S3 includes:
centrifuging a fermentation broth of the cultured bacterial cells to obtain bacterial sludge, washing the bacterial sludge, and mixing the washed bacterial sludge with a protective agent to obtain a bacterial suspension; and
freeze-drying the bacterial suspension to obtain the freeze-dried preparation.

In the present disclosure, the determining a survival rate of bacterial cells in the freeze-dried preparation in step S3 includes: determining activity of β-galactosidase, and determining activity of Na⁺-K⁺-ATPase.

The present disclosure further provides an application of the preparation method for a high-activity freeze-dried Bifidobacterium preparation based on pH regulation mentioned above in preparation of freeze-dried Bifidobacterium powder.

The present disclosure has the following beneficial effects:

The present disclosure provides the preparation method for a high-activity freeze-dried Bifidobacterium preparation based on pH regulation. The preparation method is used for influence evaluation of subsequent bacterial cell freeze drying and storage.

According to the present disclosure, by determining the influence of pH on the growth activity of the Bifidobacterium breve during culture and the influence of pH on the survival rate of the freeze-dried bacterial cells, it is found that low pH culture enhances the stability of the cell wall and membrane, thereby improving the resistance ability of the bacterial cells to freeze-drying and storage environment stress, and improving the number and activity of the bacterial cells.

The present disclosure establishes the preparation method for a high-activity freeze-dried Bifidobacterium preparation based on pH regulation, which improves the drying and storage stress resistance of the bacterial cells in a later phase, thereby providing a basis for preparing the freeze-dried Bifidobacterium powder.

The technical solutions of the present disclosure will be further described in detail in combination with the accompanying drawings and the examples.

### BRIEF DESCRIPTION OF THE DRAWINGS

FIG. 1 shows result graphs of the influence of culture pH on growth of Bifidobacterium breve B2798,
where A in FIG. 1 is a graph showing the influence of pH values on the bacterial cell density, B in FIG. 1 is a change graph showing the influence of pH values on the viable count, and C in FIG. 1 is a graph showing the influence of pH values on the maximum specific growth rate;

FIG. 2 shows graphs of the influence of pH values on the bacterial cell area of Bifidobacterium breve B2798 at various culture phases,
where S represents an average bacterial cell area in μm², n represents a sample volume, and R² represents a fitting regression coefficient;

FIG. 3 is a graph showing the influence of pH values on the freeze-drying survival rate of bacterial cells,
where different letters indicate significant differences between data (P<0.05);

FIG. 4 is a standard curve determined with reference to a β-galactosidase activity assay kit;

FIG. 5 shows the activity of β-galactosidase of Bifidobacterium breve B2798 before and after freeze drying,
where different letters indicate significant differences between data (P<0.05);

FIG. 6 shows graphs showing the relationship between β-galactosidase and the freeze-drying survival rate,
where A in FIG. 6 shows the extracellular β-galactosidase content of Bifidobacterium breve B2798 after freeze drying under different pH and different culture phases, B in FIG. 6 shows the survival rate of the Bifidobacterium breve B2798 after freeze drying during culture at the pH of 4.30, C in FIG. 6 shows the survival rate of the Bifidobacterium breve B2798 after freeze drying during culture at the pH of 5.30, D in FIG. 6 shows the survival rate of the Bifidobacterium breve B2798 after freeze drying during culture at the pH of 6.30, and in the figures, different letters indicate significant differences between data (P<0.05);

FIG. 7 is a standard curve graph determined with reference to a Na⁺-K⁺-ATPase activity assay kit; and

FIG. 8 is a graph showing the activity of Na⁺-K⁺-ATPase of Bifidobacterium breve B2798 before and after freeze drying,
where different capital letters indicate significant differences before and after freeze drying (P<0.05), and different lowercase letters indicate significant differences at different phases (P<0.05).

### DETAILED DESCRIPTIONS OF THE EMBODIMENTS

The present disclosure will be further described below with reference to the accompanying drawings and the examples. Unless otherwise defined, technical or scientific terms used in the present disclosure shall have the ordinary meaning understood by those of ordinary skill in the art to which the present disclosure pertains. The features mentioned above or the features mentioned in the particular examples of the present disclosure may be combined arbitrarily, and these particular examples are only used for illustrating the present disclosure and are not intended to limit the scope of the present disclosure.

Bifidobacterium breve B2798 involved in the examples is a disclosed strain, and is currently preserved in China General Microbiological Culture Collection Center, with the preservation number of CGMCC No. 22765, and the preservation address of No. 3, Yard 1, Beichen West road, Chaoyang District, Beijing City.

Modified MRS liquid culture medium: 10.0 g of peptone (animal origin), 8.0 g of beef extract powder, 4.0 g of yeast extract powder, 20.0 g of glucose, 1.0 mL of Tween-80, 2.0 g of dipotassium hydrogen phosphate, 5.0 g of sodium acetate, 2.0 g of triamine citrate, 0.05 g of manganese sulfate, 0.2 g of magnesium sulfate, 0.5 g of L-cysteine hydrochloride, and 1 L of distilled water; and pH is adjusted to 6.20±0.02, and sterilization is performed at 121° C. for 15 min.

Modified MRS solid culture medium: 15 g/L agar is added to the modified MRS liquid culture medium, pH is adjusted to 6.20±0.02, and sterilization is performed at 121° C. for 15 min.

### Example 1

A strain of B2798 frozen at −80° C. was inoculated in a modified MRS culture medium, placed in a constant temperature incubator, and subjected to anaerobic culture for 24 h at a constant temperature of 37.0° C. The bacterial cell vitality was recovered. Subculture was performed twice with a pipette at an inoculation amount of 2% (V/V). A third generation seed solution was inoculated in a fermentation culture medium (2 L) with an inoculation amount of 2% (V/V) by using a measuring cylinder, placed in a bioreactor for anaerobic culture at a constant temperature of 37.0° C., and fermented naturally to satisfying pH being 4.30, 4.80, 5.30, 5.80, 6.30, and 6.80. A NaOH solution at a mass concentration of 25% was used for fed-addition. Under constant control, culture was performed at different pH respectively to a logarithmic phase, a stationary phase and a death phase, thereby obtaining the bacterial cells at different pH and different growth phases.

Determination of viable count, density and maximum specific growth rate:

Samples (fermentation broths) were taken from bacterial cells at different pH and different growth phases, and the viable count was determined by a plate counting method. The processes were as follows:

The fermentation broth after sampling was diluted with sterile saline with 15 g/L tryptone until an appropriate concentration (the sample was an original resuspension at an initial phase of culture, and the sample was diluted to 10⁻¹-10⁻² at the middle and later phases, where a dilution factor in an experiment depended on a specific sample solution concentration) was achieved. A pipette was used for sucking 1 mL of bacterial suspension, and the bacterial suspension was injected into a sterile culture dish. Then, an appropriate amount of modified MRS solid culture medium was poured into the culture dish by a pouring method. After even shaking and solidification, the sample was placed in an environment at 37.0° C. for inverted anaerobic culture for 72.0 h. Three replicates were set for each sample, and the counting unit of the viable bacteria was CFU/mL.

The Bifidobacterium breve B2798 was sampled regularly in the culture process, the sample was diluted to a suitable concentration (0.20≤A=600 nm≤0.80), and 200 μL of fermentation broth was sucked by a pipette to determine its absorbance (A=600 nm) value. Three replicates were set for each sample, and the determined value was multiplied back to the dilution multiple to obtain the biomass change of the Bifidobacterium breve B2798 in the fermentation process.

According to the growth curve drawn through the bacterial cell density (A=600 nm), the maximum specific growth rate of the Bifidobacterium breve B2798 under different pH value conditions was calculated by SLogistic2 equation in Origin, and the calculation formula was as follows:

$$y = \frac{a}{1 + \frac{( a - {y}_{0} ) \times {e}^{- 4 Wmax \frac{x}{a}}}{{y}_{0}}} .$$

In the formula, x is culture time, y is a bacterial cell density, y₀ is an initial bacterial cell density, a is a maximum bacterial cell density, and Wₘₐₓ is the maximum specific growth rate.

The results of determining the viable count, the density and the maximum specific growth rate are shown in FIG. 1. It can be seen from FIG. 1 that with the decrease of pH control value, the bacterial cell density, the viable count and the maximum specific growth rate of the Bifidobacterium breve B2798 all show a trend of increasing first and then decreasing. There is no significant difference in the viable count among the experimental groups at a lag phase (P>0.05), the difference of the viable count increases significantly in the logarithmic phase, and at the end of the logarithmic phase, the viable count of the experimental group with pH of 5.30 in the culture system is (1.39±0.04)×10¹⁰ CFU/mL, which is significantly higher than that of other experimental groups (P<0.05). The maximum specific growth rate of the experimental group with pH of 5.30 in the fermentation system is (0.94±0.01) h⁻¹, which is significantly higher than that of other groups (P<0.05). There is no significant difference between the experimental groups with pH of 4.30, pH of 4.80, pH of 6.30 and pH of 6.80 respectively (P>0.05).

Therefore, the experimental groups with pH values being 4.30, 5.30 and 6.30 in the fermentation system are selected for follow-up experiments.

### Bacterial Cell Area Distribution:

Bifidobacterium breve B2798 in the culture groups with pH being 4.30, 5.30 and 6.30 respectively was sampled regularly in the culture process. 1 mL of fermentation broth was sucked, bacterial sludge was collected through centrifugation, washed with sterile PBS and precipitated, and then, resuspended to the original volume. A bacterial suspension was coated on glass slides with an inoculation ring, then, stained and fixed by using a crystal violet staining solution, and then, tested and photographed by an optical microscope BX-53. The labeled bacterial cell area was identified by Image View software. Origin202 software was used for analyzing and fitting the bacterial cell area distribution in various phases under different pH conditions, analyzing the influence of different pH on the bacterial cell size of the Bifidobacterium breve B2798 during culture, and quantifying the relationship between bacterial cell division and proliferation and the bacterial cell size. Results are shown in FIG. 2.

It can be seen from FIG. 2 that under different pH conditions, the average bacterial cell areas of the Bifidobacterium breve B2798 in various culture phases present normal distribution. At the beginning of the logarithmic phase, the bacterial cell area of the three experimental groups is maintained between 0.87-0.91 μm², and there is no significant difference. When pH is 4.30, the bacterial cell area is maintained between 0.83-0.96 μm², and there is no significant change in bacterial cell area at various culture phases (P>0.05). When the pH is 5.30, the bacterial cell area is maintained between 0.91-1.65 μm², which increases significantly by 81.32% in the middle of the logarithmic phase compared with that at the beginning of the logarithmic phase (P<0.05). At the end of the logarithmic phase, the bacterial cell area begins to decrease, which decreases by 17.58% compared with that at the middle of the logarithmic phase, but is still significantly higher than that at the beginning of the logarithmic phase (P<0.05). When the pH is 6.30, the bacterial cell area is maintained between 0.87-1.37 μm², which increases significantly by 57.47% at the middle of the logarithmic phase compared with that at the beginning of the logarithmic phase (P<0.05), and begins to decrease at the end of the logarithmic phase, which decreases by 13.24% compared with that at the middle of the logarithmic phase, but is still significantly higher than that at the beginning of the logarithmic phase (P<0.05). In combination with the results of FIG. 1 above, the promotion effect of the experimental group with the pH of 5.30 on the growth of the Bifidobacterium breve B2798 is significantly greater than those of the experimental groups with the pH of 6.30 and pH 4.30, indicating that the appropriate acidic environment is helpful to enhance the bacterial cell proliferation before cell division, resulting in a greater increase in bacterial cell area.

### Example 2

A strain of B2798 frozen at −80° C. was inoculated in a modified MRS culture medium, placed in a constant temperature incubator, and subjected to anaerobic culture for 24 h at a constant temperature of 37.0° C. The bacterial cell vitality was recovered. Subculture was performed twice with a pipette at an inoculation amount of 2% (V/V). A third generation seed solution was inoculated in a fermentation culture medium (2 L) with an inoculation amount of 2% (V/V) by using a measuring cylinder, placed in a bioreactor for anaerobic culture at a constant temperature of 37.0° C., and fermented naturally to satisfying pH being 4.30, 5.30 and 6.30. A NaOH solution at a mass concentration of 25% was used for fed-addition. Under constant control, culture was performed at different pH respectively to a logarithmic phase, a stationary phase and a death phase, thereby obtaining the bacterial cells at the logarithmic phase, the stationary phase and the death phase under the pH being 4.30, 5.30 and 6.30.

The fermentation broth of the cultured bacterial cells was centrifuged with a low-speed centrifuge at 4° C. and 4000 rpm for 15 min, supernatant was discarded, bacterial sludge was washed with sterile PBS three times, and the washed bacterial sludge was collected and then mixed with a protective agent evenly at a ratio of 1:1.2 to prepare a bacterial suspension for later use.

The prepared bacterial suspension was packaged into penicillin bottles and placed into a freeze-drying machine for vacuum freeze drying to obtain a freeze-dried preparation.

Centrifugation sampling was performed at different phases of high-density culture of the Bifidobacterium breve B2798 for vacuum freeze drying, and the change of the survival rate of strains in the freeze-dried preparations at the different growth phases was monitored. Results are shown in FIG. 3 below. It can be seen from FIG. 3 that when the pH of the culture system is 4.30, the freeze-drying survival rate of the Bifidobacterium breve B2798 sampled at different phases is significantly higher than that of the fermentation system with the pH being 5.30 and 6.30. The freeze-drying survival rate of the bacterial cells sampled at the stationary phase is the highest in the culture systems with different pH. The lower the pH value of the fermentation system, the higher the freeze-drying survival rate.

### Example 3

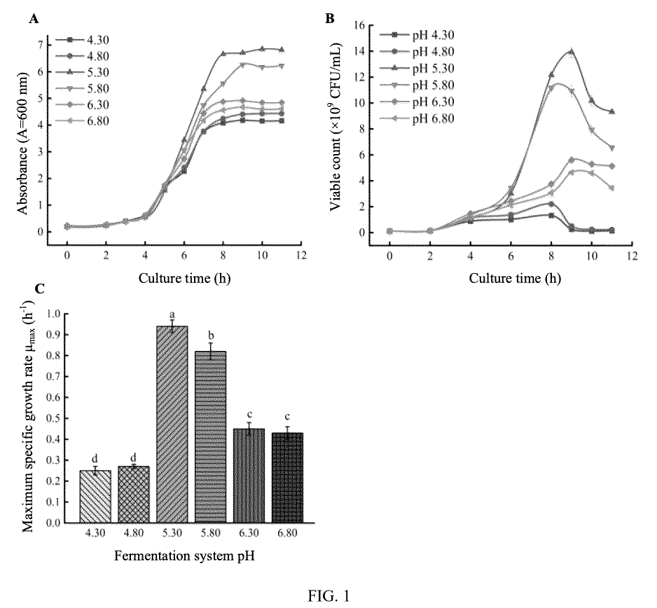

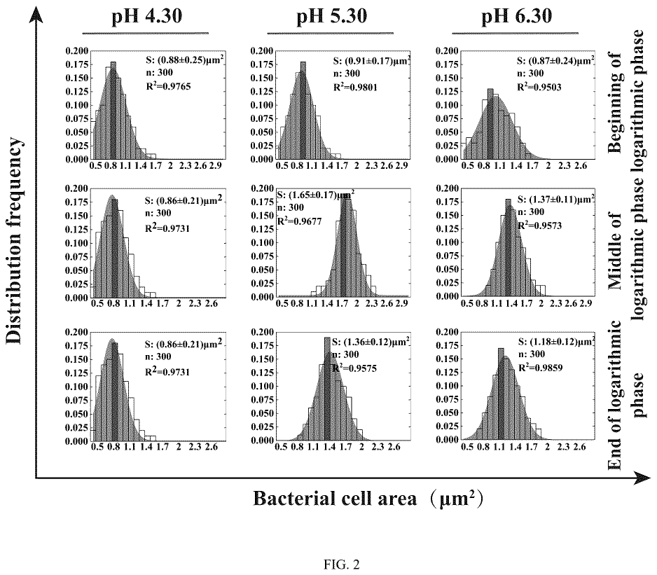

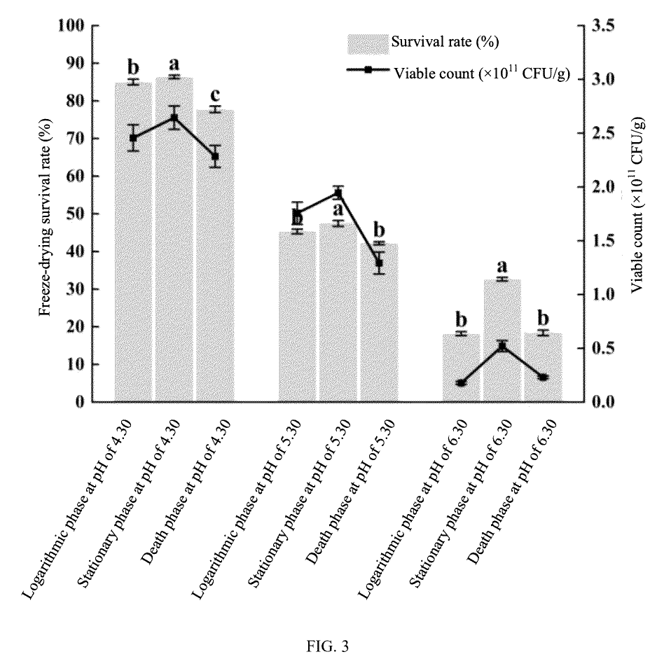

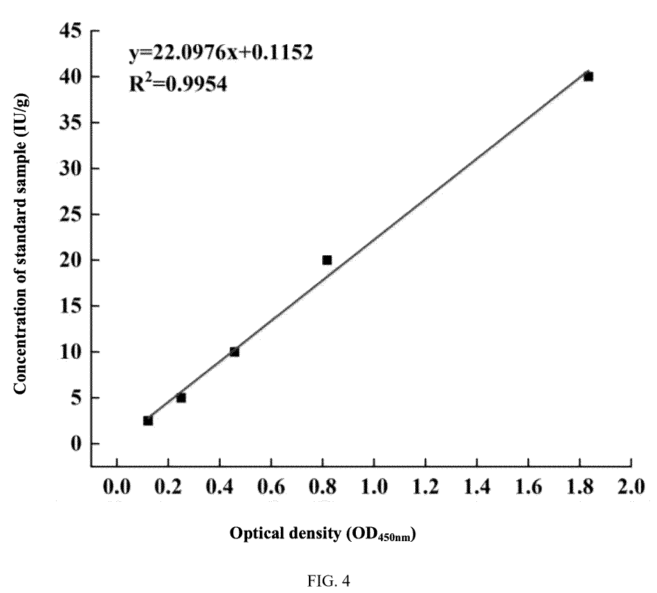

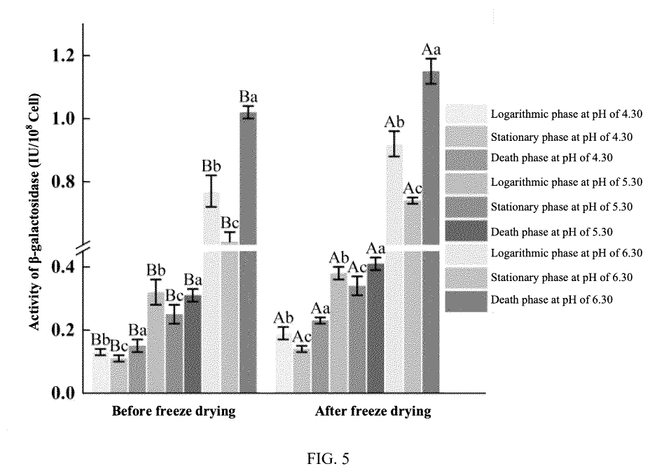

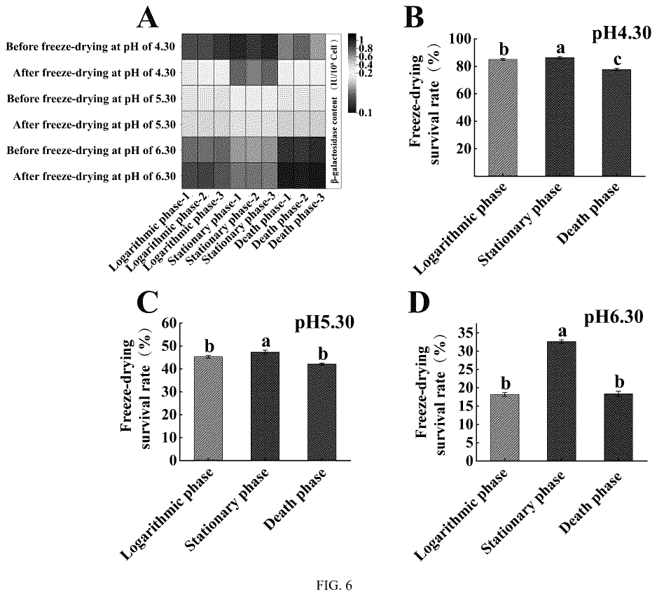

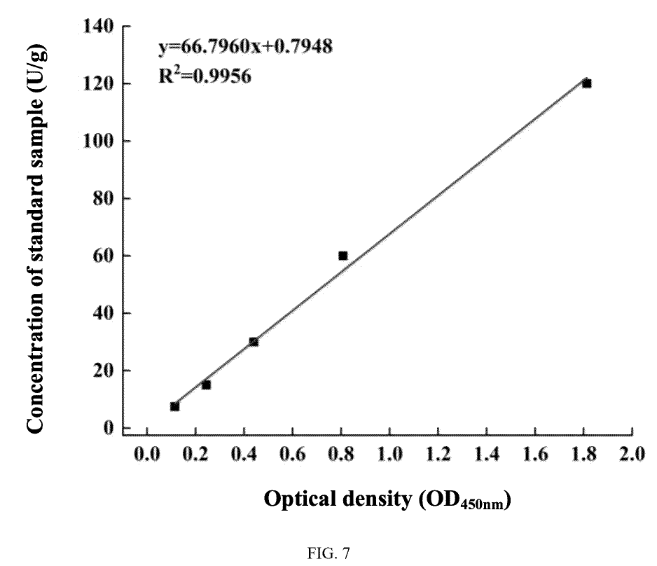

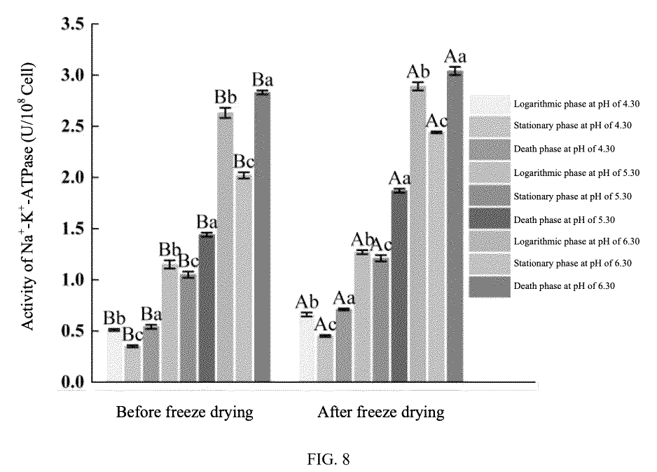

The activity of extracellular β-galactosidase of the bacterial sludge and bacterial powder of the Bifidobacterium breve B2798 was determined according to the experimental procedure of a β-galactosidase activity assay kit, and three groups of biological parallel samples were set up for data analysis. The determined standard curve is shown in FIG. 4, and the activity of β-galactosidase of the Bifidobacterium breve B2798 before and after freeze drying is shown in FIG. 5. As can be seen from FIG. 5, the activity of the extracellular β-galactosidase after freeze drying significantly increases compared with that before freeze-drying, which indicates that freeze-drying will cause damage to bacterial cell membrane and intracellular β-galactosidase will be released extracellularly. The increase of the activity of the extracellular β-galactosidase at the pH being 4.30 is smaller than that of the other two groups, indicating that low pH culture can better prevent cell membrane rupture during vacuum freeze drying. Compared with the logarithmic phase and the death phase, the activity of the extracellular β-galactosidase in the stationary phase increases least, indicating that the strain harvested in the stationary phase has stronger activity and higher resistance to a low temperature environment.

The relationship between β-galactosidase and the freeze-drying survival rate is shown in FIG. 6. At the logarithmic phase, the activity of the extracellular β-galactosidase of the Bifidobacterium breve B2798 after vacuum freeze drying is low, and in this case, the freeze-drying survival rate of the bacterial cells is at a medium level. Compared with the other two pH conditions, the freeze-drying survival rate of the bacterial cells is the highest (84.98%) at the pH of 4.30. This phenomenon indicates that the cell membrane damage degree of the freeze-dried bacterial cells cultured at the pH of 4.30 is lower.

During the stationary phase, the freeze-drying survival rate and the activity of β-galactosidase of the bacterial cells change significantly. The freeze-drying survival rate of the bacterial cells in the stationary phase is significantly higher than those in the logarithmic phase and the death phase (P<0.05). The activity of the extracellular β-galactosidase of the bacterial cells is significantly lower than those in the logarithmic phase and the death phase (P<0.05). The results show that in the stationary phase, the lower the cell membrane damage degree of the bacterial cells, the better the cell activity.

In the death phase, the freeze-drying survival rate of the Bifidobacterium breve B2798 decreases significantly, and the activity of the extracellular β-galactosidase increases significantly. Compared with the other two pH groups, the freeze-drying survival rate of the bacterial cells still keeps a relatively high level (77.73%) at the pH of 4.30. An increase of the enzyme activity is the most significant at the pH being 6.30, and the freeze-drying survival rate decreases to the lowest (18.38%), indicating that the freezing resistance of the bacterial cells decreases under neutral conditions for a long time, which leads to serious impact on the freeze-drying survival rate.

### Example 4

Pretreatment was performed on the samples according to the experimental procedure of an enzyme activity assay kit. The activity of Na⁺-K⁺-ATPase of the bacterial sludge and bacterial powder of the Bifidobacterium breve B2798 was determined, and three groups of biological parallel samples were set up for data analysis. The standard curve is shown in FIG. 7, and the activity of the Na⁺-K⁺-ATPase of the Bifidobacterium breve B2798 before and after freeze drying is shown in FIG. 8.

It can be seen from FIG. 8 that the activity of the Na⁺-K⁺-ATPase increases significantly after vacuum freeze drying, indicating that the vacuum freeze drying makes the cell membrane damaged. The increase of the enzyme activity of the bacterial cells is the smallest when culture is performed at the pH of 4.30, which may be due to the stress reaction induced by a low pH environment, which enhances the stability of the cell membrane and improves the freeze-drying survival rate of the bacterial cells. The increase of the enzyme activity of extracellular bacterial cells is the most when culture is performed at the pH of 6.30, which indicates that the cell membrane is seriously damaged when the culture is performed at the pH of 6.30.

Finally, it should be noted that the above-mentioned examples are merely intended for description of the technical solutions of the present disclosure rather than limitation of the present disclosure. Although the present disclosure is described in detail with reference to the preferred examples, those of ordinary skill in the art should understand that they may still make modifications or equivalent replacements to the technical solutions of the present disclosure. The technologies provided by the present disclosure are applicable to the Bifidobacterium, including the Bifidobacterium breve B2798, Bifidobacterium animalis subsp. lactis Probio-M8, Bifidobacterium animalis subsp. lactis V9, Bifidobacterium longum subsp. infantis B8762, etc. These modifications or equivalent replacements should not make the modified technical solutions depart from the spirit and scope of the technical solutions of the present disclosure.

What is claimed is:

1. A preparation method for high-activity freeze-dried Bifidobacterium comprising the following steps:
S1. activating a strain of Bifidobacterium breve to obtain a third generation seed solution, inoculating the third generation seed solution in a modified MRS culture medium, and culturing the Bifidobacterium breve through dynamic pH regulation;
S2. testing growth activity of bacterial cells under different pH and different growth phases; and
S3. freeze-drying the cultured bacterial cells to obtain a freeze-dried preparation, determining a survival rate of bacterial cells in the freeze-dried preparation, and screening out a culture system with the highest survival rate of bacterial cells.

2. The preparation method for high-activity freeze-dried Bifidobacterium according to claim 1, wherein an activation temperature in S1 is 36-38° C., and activation time is 23-25 h.

3. The preparation method for high-activity freeze-dried Bifidobacterium according to claim 1, wherein different growth phases in S2 comprise a logarithmic phase, a stationary phase and a death phase.

4. The preparation method for high-activity freeze-dried Bifidobacterium according to claim 1, wherein the testing growth activity of bacterial cells under different pH and different growth phases comprises testing a viable count, a density, a maximum specific growth rate and bacterial cell area distribution of the bacterial cells under different pH and different growth phases; and
a formula for calculating the maximum specific growth rate is:
$$y = \frac{a}{1 + \frac{( a - {y}_{0} ) \times {e}^{- 4 Wmax \frac{x}{a}}}{{y}_{0}}} ,$$
in the formula, x is culture time, y is a bacterial cell density, y₀ is an initial bacterial cell density, a is a maximum bacterial cell density, and Wₘₐₓ is the maximum specific growth rate.

5. The preparation method for high-activity freeze-dried Bifidobacterium according to claim 1, wherein a freeze-drying process in S3 comprises:
centrifuging a fermentation broth of the cultured bacterial cells to obtain bacterial sludge, washing the bacterial sludge, and mixing the washed bacterial sludge with a protective agent to obtain a bacterial suspension; and
freeze-drying the bacterial suspension to obtain the freeze-dried preparation.

6. The preparation method for high-activity freeze-dried Bifidobacterium according to claim 1, wherein the determining a survival rate of bacterial cells in the freeze-dried preparation in step S3 comprises: determining activity of β-galactosidase, and determining activity of Na⁺-K⁺-ATPase.

7. The preparation method for high-activity freeze-dried Bifidobacterium according to claim 1, wherein the preparation is formulated as a powder.
